(19) United States
(12) Patent Application Publication
(10) Pub. No.: US 2019/0383140 A1
(43) Pub. Date: Dec. 19, 2019

(54) METHOD AND DEVICE FOR PREDICTING CHANGE IN WATER CUT RISING RATE IN WATER-DRIVE OIL RESERVOIR

(71) Applicant: PetroChina Company Limited, Beijing (CN)

(72) Inventors: Libing FU, Beijing City (CN); Jun NI, Beijing City (CN); Xuanran LI, Beijing City (CN); Tao WANG, Beijing City (CN); Lun ZHAO, Beijing City (CN); Zifei FAN, Beijing City (CN)

(21) Appl. No.: 16/255,016

(22) Filed: Jan. 23, 2019

(30) Foreign Application Priority Data
Jun. 14, 2018 (CN) 2018106119031

(51) Int. Cl. E21B 49/08; E21B 43/20; G01N 33/28

(52) U.S. Cl. CPC E21B 49/08; E21B 43/20; E21B 2049/085; G01N 33/2823; G01N 33/2847

(57) ABSTRACT

The present invention provides a method and a device for predicting the change in the water cut rising rate of a water-drive oil reservoir. The method comprises: determining the actual water cut rising rates and water cuts of the oil reservoir, plotting the scatter plot of the actual water cut rising rates and water cuts of the oil reservoir; fitting the scatter plot of the actual water cut rising rates and water cuts of the oil reservoir to a relationship between the water cut rising rate and the water cut, to obtain the initial water cut of the oil reservoir, the degree of recovery of crude oil when the water cut of the oil reservoir is the initial water cut, the ultimate recovery of crude oil when the water cut of the oil reservoir is the water cut limit; and determining the law of change in the water cut rising rate with respect to the degree of recovery and the change in the water cut rising rate in the water-drive oil reservoir. The invention also provides a device for predicting the change in the water cut rising rate of the water-drive oil reservoir. The method and device of the present invention can predict the law of change in the water cut rising rate more correctly by considering the actual oilfield production data.

### TECHNICAL FIELD

The invention relates to a method for predicting the change in the water cut rising rate in an oil reservoir based on dynamic data from oilfield production, which belongs to the technical field of oil reservoir development.

### BACKGROUND

The water cut rising rate and the degree of recovery are important production indicators for oilfield development, and their changes reflect the effect of water-driving development of an oilfield to a certain extent. Through the statistics of the actual production data from an oilfield, it is found that there is a certain relationship between the water cut rising rate and the degree of recovery of a water-drive oil reservoir. The specific relationship is a comprehensive reflection of the law of oil and water flows under a joint action of various factors in oilfield development. The relationship between the water cut rising rate and the degree of recovery can be used to determine the change in the water cut rising rate with respect to the degree of recovery. This relationship depends not only on the oil reservoir parameters such as the reservoir heterogeneity, the nature of fluid, the size of water body, and the fluid distribution in the oil reservoir, but also on human factors such as development well patterns, developing methods, and work regulations. Therefore, even for oilfields with the same oil and water properties, the relationship between the water cut rising rate and the degree of recovery is not always the same. In order to reasonably analyze and evaluate the development effect and development degree of oilfields, and accordingly to plan development strategies and oilfield production and guide oilfield development more effectively, it is necessary to determine a reasonable relationship between the water cut rising rate and the degree of recovery.

In traditional technologies for calculating the water cut rising rate, the relationship between the permeability ratio and water saturation is calculated using the processing method of exponential formula

$$\frac{{k}_{ro}}{{k}_{rw}} = {ae}^{- {bS}_{w}}$$

based on the data of oil-water dual-phase permeability and water saturation obtained in a laboratory; and the changes in water cut and in water cut rising rate are predicted by using the fractional flow equation, and then the water-driving effect is evaluated and the indicators of development are predicted. However, the calculated results significantly deviate from the actual data near the two endpoints of the irreducible water saturation and residual oil saturation, and this deviation will remain during the calculation and will be substituted in subsequent analysis of the oil reservoir, having an adverse impact on dynamic analysis and planning of the oil reservoir. In particular, the characterization method for the above relative permeability curve has the following disadvantages: (i) the change in oilfield development indexes during the low water cut stage is inconsistent with the water-drive characteristic curve, the relative permeability ratio between oil and water is not in a linear relationship with water saturation index, and the water cut in field production often rises faster; (ii) the relative permeability curve under a high level of water injection shows a staged nonlinear characteristic. In this case, the relative permeability ratio between oil and water has little effect on the water cut in actual production. At this stage the requirement for the accuracy of characterization of the relative permeability curve is not strict, and the change in oilfield development indexes is consistent with the characteristics of a water-drive curve; (iii) the seepage flow characteristics of the oil reservoir in the ultra-high water cut stage change, and the water-drive characteristic curve shows an upturn; the relative permeability ratio between oil and water is no longer completely linear with the water saturation index, and the linear relationship only applies to the middle section of the relative permeability curve and cannot characterize the complete relative permeability curve.

In 2014, Tao Ziqiang et al., (CN application number 201410095426.X) obtained a relationship between the relative permeability ratio between oil and water and the water saturation based on a core displacement experiment, in which the relationship between the water cut rising rate and the degree of recovery was obtained by the power method:

$$\frac{\partial {f}_{w}}{\partial R} = \frac{( 1 - {S}_{wi} ) ( 1 - {S}_{or} - {S}_{wi} ) \frac{ab}{{μ}_{r}} \frac{{[ ( 1 - {S}_{wi} ) R ]}^{- b - 1}}{{[ 1 - {S}_{or} - {S}_{wi} - ( 1 - {S}_{wi} ) R ]}^{- b + 1}}}{{\{ 1 + \frac{1}{{μ}_{r}} {a [ \frac{( 1 - {S}_{wi} ) R}{1 - {S}_{or} - {S}_{wi} - ( 1 - {S}_{wi} ) R} ]}^{- b} \}}^{2}}$$

wherein f_{w} is the water cut of oil reservoir, R is the degree of recovery of oil reservoir, Sₒᵣ is the residual oil saturation, S_{wi} is the irreducible water saturation, μᵣ is the viscosity ratio between oil and water, a and b are constants obtained by a regression fit to the oil and water relative permeability curve.

The above method proposes that as long as the oil and water relative permeability curve is known, the change in the water cut rising rate of an oilfield can be predicted. However, the actual change in water cut of an oilfield is not only related to the oil and water relative permeability curve, but also considerably related to the well pattern and development mode of the oilfield. The above method cannot well reflect the actual production characteristics of an oilfield, and cannot work well in actual evaluation of effects and prediction of indexes for water-driving development of an oilfield; its practicability is poor.

### SUMMARY OF INVENTION

In order to solve the above technical problems, an objective of the present invention is to provide a method for predicting the law of change in the water cut rising rate based on actual production data from an oil reservoir.

In order to achieve the above technical objective, the present invention provides a method for predicting the change in the water cut rising rate in a water-drive oil reservoir, the method comprising the following steps:

determining the actual water cut rising rates and water cuts of the oil reservoir, and plotting a scatter plot of the actual water cut rising rates and water cuts for the oil reservoir;

fitting the scatter plot of the actual water cut rising rates and water cuts of the oil reservoir to a relationship between the water cut rising rate and the water cut, to obtain the initial water cut of the oil reservoir, the degree of recovery of crude oil when the water cut of the oil reservoir is the initial water cut, and the ultimate recovery of crude oil when the water cut of the oil reservoir is the water cut limit; and

deriving the law of change in the water cut rising rate with respect to the degree of recovery from the initial water cut of the oil reservoir, the degree of recovery of crude oil when the water cut of the oil reservoir is the initial water cut, and the ultimate recovery of crude oil when the water cut of the oil reservoir is the water cut limit, to determine the change in the water cut rising rate in the water-drive oil reservoir.

In the method according to the present invention, in the fitting step, a scatter plot of the actual degrees of recovery and the water cuts is fitted to a relationship between the degree of recovery and the water cut by nonlinear regression.

In the method according to the present invention, preferably, the relationship between the water cut rising rate and the water cut used is as follows:

$$\frac{{df}_{w}}{dR} = {cf}_{w} ( 1 - {f}_{w} ) \frac{log ( \frac{{f}_{wL}}{1 - {f}_{wL}} ) - log ( \frac{{f}_{w 0}}{1 - {f}_{w 0}} )}{{E}_{R} - {R}_{0}} ;$$

wherein

$$\frac{{df}_{w}}{dR}$$

is the water cut rising rate;

f_{w} is the water cut of the oil reservoir;

f_{w0} is the initial water cut of the oil reservoir;

f_{wL} is the water cut limit of the oil reservoir, usually being 0.98;

R is the degree of recovery of the oil reservoir;

R₀ is the degree of recovery of crude oil when the water cut of the oil reservoir is f_{w0};

E_{R} is the ultimate recovery of crude oil when the water cut of the oil reservoir is the water cut limit f_{wL};

c=In (10).

In the method according to the present invention, preferably, the law of change in the water cut rising rate with respect to the degree of recovery is determined according to the following equation:

$$\frac{{df}_{w}}{dR} = c \frac{\begin{matrix}\frac{log ( \frac{{f}_{wL}}{1 - {f}_{wL}} ) - log ( \frac{{f}_{w 0}}{1 - {f}_{w 0}} )}{{E}_{R} - {R}_{0}} \times \\ {10}^{\frac{log ( \frac{{f}_{w 0}}{1 - {f}_{w 0}} ) ( R - {E}_{R} ) + log ( \frac{{f}_{wL}}{1 - {f}_{wL}} ) ( {R}_{0} - R )}{{E}_{R} - {R}_{0}}}\end{matrix}}{{\{ 1 + {10}^{\frac{log ( \frac{{f}_{w 0}}{1 - {f}_{w 0}} ) ( R - {E}_{R} ) + log ( \frac{{f}_{wL}}{1 - {f}_{wL}} ) ( {R}_{0} - R )}{{E}_{R} - {R}_{0}}} \}}^{2}} ,$$

wherein

$$\frac{{df}_{w}}{dR}$$

is the water cut rising rate;

f_{w} is the water cut of the oil reservoir;

f_{w0} is the initial water cut of the oil reservoir;

f_{wL} is the water cut limit of the oil reservoir, usually being 0.98;

R is the degree of recovery of the oil reservoir;

R₀ is the degree of recovery of crude oil when the water cut of the oil reservoir is f_{w0};

E_{R} is the ultimate recovery of crude oil when the water cut of the oil reservoir is the water cut limit f_{wL};

c=In(10).

The method for predicting the change in the water cut rising rate of a water-drive oil reservoir according to the present invention is based on the actual production data of the oil reservoir, contemplates the relationship between the water cut and the water cut rising rate in actual production characteristic of the oil reservoir, and uses a nonlinear regression mathematical fitting method to obtain the initial water cut, the degree of recovery, the ultimate recovery and the corresponding relationship between the water cut and the water cut rising rate that characterize the actual water-driving of the oil reservoir, and further to obtain the theoretical relationship reflecting the change in the water cut rising rate in the oilfield.

The method for predicting the change in the water cut rising rate of a water-drive oil reservoir according to the invention can more correctly predict the law of change in the water cut rising rate, and is especially suitable for prediction of the change in the water cut rising rate of an oil reservoir satisfying the type A water-drive production curve.

An embodiment of the present invention also provides a method for predicting the dynamic state of an oil reservoir during water-driving development, the method comprising the steps of the above method for predicting the change in the water cut rising rate of a water-drive oil reservoir according to the present invention.

The above method for predicting according to the present invention preferably comprises the following steps:

obtaining the law of change in the water cut rising rate with respect to the degree of recovery by the method for predicting the change in the water cut rising rate of the water-drive oil reservoir according to the present invention; and

comparing the actual data of the relationship between the water cut rising rate and the degree of recovery with the law of change in the water cut rising rate with respect to the degree of recovery, and then analyzing the effect of the water-driving development of the oil reservoir.

In the method for predicting the dynamic state of an oil reservoir during water-driving development according to the present invention, if the actual values of the water cut rising rate are larger than the theoretical water cut rising rates, the development effect is poor; if the actual values of the water cut rising rate are equal to the theoretical water cut rising rates, the development effect is good; and if the actual values of the water cut rising rates are smaller than the theoretical water cut rising rates, the development effect is excellent.

The method for predicting the dynamic state of an oil reservoir during water-driving development according to the present invention comprises obtaining the law of change in the water cut rising rate with respect to the degree of recovery by the method for predicting the change in the water cut rising rate of a water-drive oil reservoir according to the present invention, and comparing it with the actual values of the oil reservoir to reasonably analyze and evaluate the water-driving effect and development characteristics of the oil reservoir, and accordingly plan development strategies and oilfield production for the oil reservoir and effectively guide the remaining oil tapping potential and oil reservoir development.

A further embodiment of the invention provides a device for predicting the change in the water cut rising rate of a water-drive oil reservoir, the device comprising:

an actual-data plotting module, which is configured to determine the actual water cut rising rates and water cuts of the oil reservoir, and plot a scatter plot of the actual water cut rising rates and water cuts of the oil reservoir;

a parameter determining module, which is configured to fit the scatter plot of the actual water cut rising rates and water cuts of the oil reservoir to a relationship between the water cut rising rate and the water cut, and to obtain the initial water cut of the oil reservoir, the degree of recovery of crude oil when the water cut of the oil reservoir is the initial water cut, and the ultimate recovery of crude oil when the water cut of the oil reservoir is the water cut limit;

a determining module, which is configured to derive the law of change in the water cut rising rate with respect to the degree of recovery from the initial water cut of the oil reservoir, the degree of recovery of crude oil when the water cut of the oil reservoir is the initial water cut, and the ultimate recovery of crude oil when the water cut of the oil reservoir is the water cut limit, and to determine the change in the water cut rising rate in the water-drive oil reservoir.

In the device according to the invention, preferably, the relationship between the water cut rising rate and the water cut is as follows:

$$\frac{{df}_{w}}{dR} = {cf}_{w} ( 1 - {f}_{w} ) \frac{log ( \frac{{f}_{wL}}{1 - {f}_{wL}} ) - log ( \frac{{f}_{w 0}}{1 - {f}_{w 0}} )}{{E}_{R} - {R}_{0}} ;$$

wherein

$$\frac{{df}_{w}}{dR}$$

is the water cut rising rate;

f_{w} is the water cut of the oil reservoir;

f_{w0} is the initial water cut of the oil reservoir;

f_{wL} is the water cut limit of the oil reservoir, usually being 0.98;

R is the degree of recovery of the oil reservoir;

R₀ is the degree of recovery of crude oil when the water cut of the oil reservoir is f_{w0};

E_{R} is the ultimate recovery of crude oil when the water cut of the oil reservoir is water cut limit f_{wL};

c=In (10).

In the device according to the invention, preferably, the law of change in the water cut rising rate with respect to the degree of recovery is determined according to the following equation:

$$\frac{{df}_{w}}{dR} = c \frac{\begin{matrix}\frac{log ( \frac{{f}_{wL}}{1 - {f}_{w L}} ) - log ( \frac{{f}_{w 0}}{1 - {f}_{w 0}} )}{{E}_{R} - {R}_{0}} \times \\ {10}^{\frac{log ( \frac{{f}_{w 0}}{1 - {f}_{w 0}} ) ( R - {E}_{R} ) + log ( \frac{{f}_{wL}}{1 - {f}_{w L}} ) ( {R}_{0} - R )}{{E}_{R} - {R}_{0}}}\end{matrix}}{{\{ 1 + {10}^{\frac{log ( \frac{{f}_{w 0}}{1 - {f}_{w 0}} ) ( R - {E}_{R} ) + log ( \frac{{f}_{w L}}{1 - {f}_{w L}} ) ( {R}_{0} - R )}{{E}_{R} - {R}_{0}}} \}}^{2}} , \qquad$$

wherein

$$\frac{{df}_{w}}{dR}$$

is water cut rising rate;

f_{w} is the water cut of the oil reservoir;

f_{w0} is the initial water cut of the oil reservoir;

f_{wL} is the water cut limit of the oil reservoir, usually being 0.98;

R is the degree of recovery of the oil reservoir;

R₀ is the degree of recovery of crude oil when the water cut of the oil reservoir is f_{w0};

E_{R} is the ultimate recovery of crude oil when the water cut of the oil reservoir is water cut limit f_{wL};

c=In(10).

The device for predicting the change in the water cut rising rate of a water-drive oil reservoir according to the present invention is based on the actual production data of the oil reservoir, contemplates the relationship between the water cut and the water cut rising rate in actual production characteristic of the oil reservoir, and uses a nonlinear regression mathematical fitting method to obtain the initial water cut, the degree of recovery, the ultimate recovery and the corresponding relationship between the water cut and the degree of recovery that characterize the actual water-driving of the oil reservoir, and further to obtain the theoretical relationship reflecting the change in the water cut rising rate in the oil reservoir.

The device for predicting the change in the water cut rising rate of a water-drive oil reservoir according to the present invention can more correctly predict the law of change in the water cut rising rate, and is especially suitable for prediction of the change in the water cut rising rate of an oil reservoir satisfying the type A water-drive production curve.

The method and device for predicting the change in the water cut rising rate of a water-drive oil reservoir according to the present invention can more correctly determine the law of change in the water cut rising rate based on the actual production data of the oil reservoir. Based on the theories of oil reservoir engineering and fluid mechanics in combination with the actual production data, a general solution for the relationship between the water cut rising rate and water cut and the degree of recovery of a water-drive oil reservoir is proposed as an equation, and a water cut rising rate curve corresponding to the actual water-driving law of oilfields is plotted, which can, theoretically and practically, more accurately explain and analyze the law characteristic of actual water-driving of oilfields and predict indicators for future development of oilfields.

The method for predicting the dynamic state of an oil reservoir during water-driving development according to the present invention, by employing the method for predicting the change in the water cut rising rate of a water-drive oil reservoir according to the present invention, can reasonably analyze and evaluate the effect of water-driving development of the oil reservoir based on actual production data of the oil reservoir, plan development strategies and production for the oil reservoir, and effectively guide the remaining oil tapping potential and oil reservoir development.

### DESCRIPTION OF DRAWINGS

FIG. 1 is a schematic view of a device for predicting the change in the water cut rising rate in a water-drive oil reservoir according to an example of the present invention;

FIG. 2 is a comparison between the actual data and a curve of a relationship between the water cut rising rate and water cut according to an example of the present invention;

FIG. 3 is a comparison between the actual data and a curve of a relationship between the water cut rising rate and the degree of recovery according to an example of the present invention.

### DETAILED DESCRIPTION OF INVENTION

In order to provide a better understanding of the technical features, objectives, and advantages of the present invention, the technical solutions of the present invention are described in detail below, but are not to be construed as limiting the scope of the invention.

### EXAMPLE 1

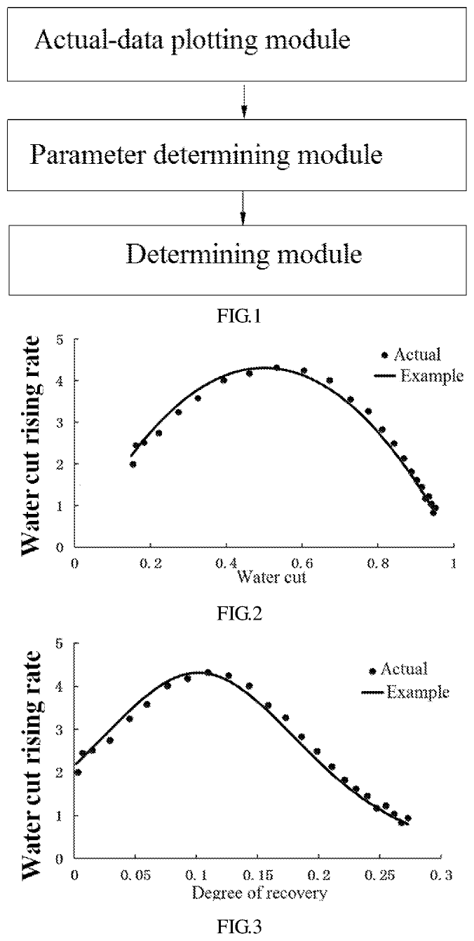

This example first provides a device for predicting the change in the water cut rising rate of a water-drive oil reservoir, which has a structure shown in FIG. 1, and may comprise:

an actual-data plotting module, which is configured to determine the actual water cut rising rates and water cuts of the oil reservoir, and plot a scatter plot of the actual water cut rising rates and water cuts of the oil reservoir;

a parameter determining module, which is configured to fit the scatter plot of the actual water cut rising rates and water cuts of the oil reservoir to a relationship between the water cut rising rate and the water cut, and to obtain the initial water cut of the oil reservoir, the degree of recovery of crude oil when the water cut of the oil reservoir is the initial water cut, and the ultimate recovery of crude oil when the water cut of the oil reservoir is the water cut limit; and

a determining module, which is configured to derive the law of change in the water cut rising rate with respect to the degree of recovery from the initial water cut of the oil reservoir, the degree of recovery of crude oil when the water cut of the oil reservoir is the initial water cut, and the ultimate recovery of crude oil when the water cut of the oil reservoir is the water cut limit, and to determine the change in the water cut rising rate in the water-drive oil reservoir.

The example also provides a method for predicting the change in the water cut rising rate in the water-drive oil reservoir, and the method may comprise the following steps.

Firstly, the geological and developing conditions of the oil reservoir were investigated, and the production data of the oilfield in the past development were obtained. According to the production data, the actual degree of recovery, water cut, and water cut rising rate of the oilfield were calculated (see Table 1). The actual water cut rising rates (in the y axis) of the oilfield were plotted versus the water cuts (in the x axis) of the oilfield (see the scatter point in FIG. 2).

TABLE 1

| Production | Cumulative oil | Cumulative water | Water | Degree of | Water cut |
|---|---|---|---|---|---|
| time/year | production/10⁴ t | production/10⁴ t | cut | recovery | rising rate |
| 1 | 5.66 | 0.10 | 0.150 | 0.0013 | |
| 2 | 14.36 | 0.22 | 0.154 | 0.0033 | 1.9954 |
| 3 | 30.34 | 1.73 | 0.163 | 0.0070 | 2.4443 |
| 4 | 64.86 | 6.12 | 0.183 | 0.0149 | 2.5145 |
| 5 | 128.10 | 15.70 | 0.223 | 0.0295 | 2.7451 |
| 6 | 196.27 | 28.99 | 0.274 | 0.0452 | 3.2469 |
| 7 | 259.40 | 53.15 | 0.326 | 0.0598 | 3.5748 |
| 8 | 331.97 | 103.78 | 0.393 | 0.0765 | 4.0069 |
| 9 | 402.78 | 176.00 | 0.461 | 0.0928 | 4.1678 |
| 10 | 475.19 | 268.77 | 0.533 | 0.1095 | 4.3154 |
| 11 | 548.78 | 378.76 | 0.605 | 0.1264 | 4.2462 |
| 12 | 622.48 | 508.57 | 0.673 | 0.1434 | 4.0043 |
| 13 | 689.66 | 671.11 | 0.728 | 0.1589 | 3.5531 |
| 14 | 752.08 | 876.57 | 0.775 | 0.1733 | 3.2679 |
| 15 | 807.46 | 1117.87 | 0.811 | 0.1861 | 2.8212 |
| 16 | 863.29 | 1417.85 | 0.843 | 0.1989 | 2.4876 |
| 17 | 916.31 | 1744.16 | 0.869 | 0.2111 | 2.1283 |
| 18 | 961.72 | 2079.29 | 0.888 | 0.2216 | 1.8159 |
| 19 | 1002.10 | 2418.27 | 0.903 | 0.2309 | 1.6122 |
| 20 | 1041.10 | 2771.89 | 0.916 | 0.2399 | 1.4467 |
| 21 | 1074.57 | 3115.38 | 0.925 | 0.2476 | 1.1670 |
| 22 | 1106.60 | 3471.50 | 0.934 | 0.2550 | 1.2195 |
| 23 | 1135.98 | 3842.26 | 0.941 | 0.2617 | 1.0340 |
| 24 | 1162.30 | 4228.46 | 0.946 | 0.2678 | 0.8245 |
| 25 | 1185.37 | 4584.76 | 0.951 | 0.2731 | 0.9406 |

Secondly, the actual data of the water cut rising rates and water cuts of the oilfield shown in FIG. 2 were fitted to a relationship between the water cut rising rate and the water cut by nonlinear regression, to obtain the initial water cut of the oil reservoir, the degree of recovery of crude oil when the water cut of the oil reservoir is the initial water cut, the ultimate recovery of crude oil when the water cut of the oil reservoir is the water cut limit;

wherein the relationship between the water cut rising rate and the water cut is as follows:

$$\frac{{df}_{w}}{dR} = {cf}_{w} ( 1 - {f}_{w} ) \frac{log ( \frac{{f}_{wL}}{1 - {f}_{wL}} ) - log ( \frac{{f}_{w 0}}{1 - {f}_{w 0}} )}{{E}_{R} - {R}_{0}} ,$$

wherein f_{w} is the water cut of the oil reservoir;

f_{w0} is the initial water cut of the oil reservoir, being 0.15;

f_{wL} is the water cut limit of the oil reservoir, being 0.98;

R is the degree of recovery of the oil reservoir;

R₀ is the degree of recovery of crude oil when the water cut of the oil reservoir is f_{w0}, being 0;

E_{R} is the ultimate recovery of crude oil when the water cut of the oil reservoir is the water cut limit f_{wL}, being 0.326.

The obtained relationship of the water cut rising rate and water cut is as follows:

$$\frac{{df}_{w}}{dR} = 17.26 {f}_{w} ( 1 - {f}_{w} ) .$$

Thirdly, based on the obtained parameters R₀(R₀=0), f_{w0} (f_{w0}=0.15) and the ultimate recovery E_{R} (E_{R}=0.326), and according to the following formula

$$\frac{{df}_{w}}{d R} = c \frac{\begin{matrix}\frac{log ( \frac{{f}_{w L}}{1 - {f}_{wL}} ) - log ( \frac{{f}_{w 0}}{1 - {f}_{w 0}} )}{{E}_{R} - {R}_{0}} \times \\ {10}^{\frac{log ( \frac{{f}_{w 0}}{1 - {f}_{w 0}} ) ( R - {E}_{R} ) + log ( \frac{{f}_{w L}}{1 - {f}_{wL}} ) ( {R}_{0} - R )}{{E}_{R} - {R}_{0}}}\end{matrix}}{{\{ 1 + {10}^{\frac{log ( \frac{{f}_{w 0}}{1 - {f}_{w 0}} ) ( R - {E}_{R} ) + log ( \frac{{f}_{wL}}{1 - {f}_{w L}} ) ( {R}_{0} - R )}{{E}_{R} - {R}_{0}}} \}}^{2}} ,$$

wherein

$$\frac{{df}_{w}}{dR}$$

is the water cut rising rate; f_{w} is the water cut of the oil reservoir; f_{w0} is the initial water cut of the oil reservoir; f_{wL} is 0.98; R is the degree of recovery of the oil reservoir; R₀ is the degree of recovery of crude oil when the water cut of the oil reservoir is f_{w0}; E_{R} is the ultimate recovery of crude oil when the water cut of the oil reservoir is the water cut limit f_{wL}, and c=ln(10);

the law of change in the water cut rising rate with respect to the degree of recovery was obtained as follows:

$$\frac{{df}_{w}}{dR} = \frac{17.25896 \times {10}^{0.75333 - 7.49547 R}}{{( 1 + {10}^{0.75333 - 7.49547 R} )}^{2}} .$$

The actual value and theoretical value of the water cut rising rates (in the y axis) were plotted versus those of the degree of recovery (in the x axis) for the oil reservoir of the present example (the scatter points in FIG. 3), and the theoretical curve of the relationship between the water cut rising rate and the degree of recovery was also drawn in the coordinate system (the solid line in FIG. 3).

The present example further provides a method for predicting the dynamic state of an oil reservoir during water-driving development, and the method for predicting may include the following steps:

comparing the actual data of the relationship between the water cut rising rate and the degree of recovery with the law of change in the water cut rising rate with respect to the degree of recovery obtained by the method above, with the result shown in FIG. 3.

From both the plot of the relationship between the water cut rising rate and the water cut and the plot of the relationship between the water cut rising rate and the degree of recovery, it can be seen that the actual data points fluctuate on or near the curve of the present example, and match the curve very well, indicating that the figures obtained by the method of the present invention can effectively reflect the true law of change in the water cut rising rate of an oilfield, can work well in analyzing the water injection development effect on an oilfield and predicting indexes therefor, and provides a more reasonable and reliable reference guidance for adjustment of the developing plan of the oilfield and the rest of steps of potential oil development.

For convenience of description, the above device is described in functionally separate units. However, the functions of the various units may be implemented in one or more pieces of software and/or hardware in implementation of the invention.

The present invention has been described with reference to the flowcharts and/or block diagrams of methods, device (system), and computer program products according to examples of the invention. It will be understood that each procedure in the flow and/or block of the flowcharts and/or block diagrams and the combinations thereof can be implemented by computer program instructions. These computer program instructions can be provided to a processor of a general purpose computer, a special purpose computer, an embedded processor, or other programmable data processing devices to produce a machine, such that the execution of the instructions by a processor of a computer or other programmable data processing devices produces a device for implementing the functions specified in one or more procedures in the flow of the flowcharts or in one or more blocks of the block diagrams.

The computer program instructions can also be stored in a computer readable memory that can direct a computer or other programmable data processing devices to operate in a particular manner, such that the instructions stored in the computer readable memory produce an manufactured product comprising an instruction means which implements the functions specified in one or more procedures in the flow of the flowcharts or in one or more blocks of the block diagrams.

These computer program instructions can also be installed in a computer or other programmable data processing devices such that a series of operational steps are performed on the computer or other programmable devices to produce a computer-implemented processing, such that execution of the instructions on the computer or other programmable devices provides steps for implementing the functions specified in one or more procedures in the flow of the flowcharts or in one or more blocks of the block diagrams.

In a typical configuration, a computing device includes one or more processors (CPUs), input/output interfaces, network interfaces, and memory.

The memory may include non-persistent memory, random access memory (RAM), and/or non-volatile memory in a computer readable medium, such as read only memory (ROM) or flash memory (flash RAM). Memory is an example of a computer readable medium.

A computer readable medium, including a permanent or non-permanent, removable or non-removable medium, may store information by any method or technology. The information can be computer readable instructions, data structures, program modules, or other data. Examples of a computer storage medium include, but are not limited to, phase change memory (PRAM), static random access memory (SRAM), dynamic random access memory (DRAM), other types of random access memory (RAM), read only memory (ROM), electrically erasable programmable read only memory (EEPROM), flash memory or other memory technology, compact disk read only memory (CD-ROM), digital versatile disk (DVD) or other optical storage, cassette magnetic tape, magnetic tape storage or other magnetic storage devices, or any other non-transmittable media, which can be used to store information accessible by a computing device. As defined herein, a computer readable medium does not include transitory media, such as modulated data signals and carrier waves.

It is also to be understood that the terms “comprise” or “include” or any other variations are intended to encompass a non-exclusive inclusion, such that a process, method, product, or device that includes a series of elements includes not only those elements but also other elements not explicitly listed, or inherent in the process, method, product, or device. Without further restrictions, the elements defined by the statement “including one . . . ” do not exclude the existence of more of the same elements in the process, method, product, or device that includes the element.

Those skilled in the art will appreciate that examples of the present invention can be provided as a method, system, or computer program product. Accordingly, the present invention may take the form of an entirely hardware embodiment, an entirely software embodiment, or a combination embodiment of software and hardware. Moreover, the present invention can take the form of a computer program product implemented on one or more computer-usable storage media (including but not limited to disk storage, CD-ROM, optical storage, etc.) including the computer implementable program code.

The invention may be described in the general context of computer-executable instructions executed by a computer, such as a program module. Generally, a program module includes routines, programs, objects, components, data structures, and the like that perform particular tasks or implement particular abstract data types. The invention may also be practiced in distributed computing environments where tasks are performed by remote processing devices that are connected via a communication network. In a distributed computing environment, program modules can be located in both local and remote computer storage media including storage devices.

The various examples in the specification are described in a progressive manner, and the same or similar parts among the various examples may be referred to each other. Each example focuses on the differences from the other examples. In particular, for the device example, since it is basically similar to the method example, the description is relatively simplified, and the relevant parts can be referred to the description of the method example.

1. A method for predicting the change in the water cut rising rate in a water-drive oil reservoir,
comprising:
determining the actual water cut rising rates and water cuts of the oil reservoir, plotting a scatter plot of the actual water cut rising rates and water cuts of the oil reservoir;
fitting the scatter plot of the actual water cut rising rates and water cuts of the oil reservoir to a relationship between the water cut rising rate and the water cut, to obtain the initial water cut of the oil reservoir, the degree of recovery of crude oil when the water cut of the oil reservoir is the initial water cut, and the ultimate recovery of crude oil when the water cut of the oil reservoir is the water cut limit; and
deriving a relationship of change in the water cut rising rate with respect to the degree of recovery from the initial water cut of the oil reservoir, the degree of recovery of crude oil when the water cut of the oil reservoir is the initial water cut, and the ultimate recovery of crude oil when the water cut of the oil reservoir is the water cut limit, to determine the change in the water cut rising rate in the water-drive oil reservoir.

2. The method according to claim 1, wherein the relationship between the water cut rising rate and the water cut is as follows:
$$\frac{{df}_{w}}{dR} = {cf}_{w} ( 1 - {f}_{w} ) \frac{log ( \frac{{f}_{wL}}{1 - {f}_{wL}} ) - log ( \frac{{f}_{w 0}}{1 - {f}_{w 0}} )}{{E}_{R} - {R}_{0}} ;$$
wherein
$$\frac{{df}_{w}}{dR}$$
is the water cut rising rate;
f_{w} is the water cut of the oil reservoir;
f_{w0} is the initial water cut of the oil reservoir;
f_{wL} is the water cut limit of the oil reservoir;
R is the degree of recovery of the oil reservoir;
R₀ is the degree of recovery of crude oil when the water cut of the oil reservoir is f_{w0};
E_{R} is the ultimate recovery of crude oil when the water cut of the oil reservoir is the water cut limit f_{wL};
c=ln(10).

3. The method according to claim 1, wherein the relationship of change in the water cut rising rate with respect to the degree of recovery is determined according to the following equation:
$$\frac{{df}_{w}}{d R} = c \frac{\begin{matrix}\frac{log ( \frac{{f}_{w L}}{1 - {f}_{wL}} ) - log ( \frac{{f}_{w 0}}{1 - {f}_{w 0}} )}{{E}_{R} - {R}_{0}} \times \\ {10}^{\frac{log ( \frac{{f}_{w 0}}{1 - {f}_{w 0}} ) ( R - {E}_{R} ) + log ( \frac{{f}_{w L}}{1 - {f}_{wL}} ) ( {R}_{0} - R )}{{E}_{R} - {R}_{0}}}\end{matrix}}{{\{ 1 + {10}^{\frac{log ( \frac{{f}_{w 0}}{1 - {f}_{w 0}} ) ( R - {E}_{R} ) + log ( \frac{{f}_{wL}}{1 - {f}_{w L}} ) ( {R}_{0} - R )}{{E}_{R} - {R}_{0}}} \}}^{2}} ,$$
wherein
$$\frac{{df}_{w}}{dR}$$
is the water cut rising rate;
f_{w} is the water cut of the oil reservoir;
f_{w0} is the initial water cut of the oil reservoir;
f_{wL} is the water cut limit of the oil reservoir;
R is the degree of recovery of the oil reservoir;
R₀ is the degree of recovery of crude oil when the water cut of the oil reservoir is f_{w0};
E_{R} is the ultimate recovery of crude oil when the water cut of the oil reservoir is the water cut limit f_{wL};
c=ln(10).

4. A method for predicting the dynamic state of an oil reservoir during water-driving development, wherein the method for predicting comprises the steps of claim 1.

5. The method for predicting according to claim 4, wherein the method comprises:
obtaining the relationship of change in the water cut rising rate with respect to the degree of recovery by the method according to claim 1; and
comparing the actual data of the relationship between the water cut rising rate and the degree of recovery with the relationship of change in the water cut rising rate with respect to the degree of recovery, and then analyzing the effect of the water-driving development of the oil reservoir.

6. A device for predicting the change in the water cut rising rate of a water-drive oil reservoir, comprising:
an actual-data plotting module, which is configured to determine the actual water cut rising rates and water cuts of the oil reservoir, and plot a scatter plot of the actual water cut rising rates and water cuts of the oil reservoir;
a parameter determining module, which is configured to fit the scatter plot of the actual water cut rising rates and water cuts of the oil reservoir to a relationship between the water cut rising rate and the water cut, and to obtain the initial water cut of the oil reservoir, the degree of recovery of crude oil when the water cut of the oil reservoir is the initial water cut, and the ultimate recovery of crude oil when the water cut of the oil reservoir is the water cut limit; and
a determining module, which is configured to derive the relationship of change in the water cut rising rate with respect to the degree of recovery from the initial water cut of the oil reservoir, the degree of recovery of crude oil when the water cut of the oil reservoir is the initial water cut, and the ultimate recovery of crude oil when the water cut of the oil reservoir is the water cut limit, and to determine the change in the water cut rising rate in the water-drive oil reservoir.

7. The device according to claim 6, wherein the relationship between the water cut rising rate and the water cut is as follows:
$$\frac{{df}_{w}}{dR} = {cf}_{w} ( 1 - {f}_{w} ) log \frac{( \frac{{f}_{wL}}{1 - {f}_{wL}} ) - log ( \frac{{f}_{w 0}}{1 - {f}_{w 0}} )}{{E}_{R} - {R}_{0}} ;$$
wherein
$$\frac{{df}_{w}}{dR}$$
is the water cut rising rate;
f_{w} is the water cut of the oil reservoir;
f_{w0} is the initial water cut of the oil reservoir;
f_{wL} is the water cut limit of the oil reservoir;
R is the degree of recovery of the oil reservoir;
R₀ is the degree of recovery of crude oil when the water cut of the oil reservoir is f_{w0};
E_{R} is the ultimate recovery of crude oil when the water cut of the oil reservoir is the water cut limit f_{wL};
c=ln(10).

8. The device according to claim 6, wherein relationship in the water cut rising rate with respect to the degree of recovery is determined according to the following equation:
$$\frac{{df}_{w}}{d R} = c \frac{\begin{matrix}\frac{log ( \frac{{f}_{w L}}{1 - {f}_{wL}} ) - log ( \frac{{f}_{w 0}}{1 - {f}_{w 0}} )}{{E}_{R} - {R}_{0}} \times \\ {10}^{\frac{log ( \frac{{f}_{w 0}}{1 - {f}_{w 0}} ) ( R - {E}_{R} ) + log ( \frac{{f}_{w L}}{1 - {f}_{wL}} ) ( {R}_{0} - R )}{{E}_{R} - {R}_{0}}}\end{matrix}}{{\{ 1 + {10}^{\frac{log ( \frac{{f}_{w 0}}{1 - {f}_{w 0}} ) ( R - {E}_{R} ) + log ( \frac{{f}_{wL}}{1 - {f}_{w L}} ) ( {R}_{0} - R )}{{E}_{R} - {R}_{0}}} \}}^{2}} ,$$
wherein
$$\frac{{df}_{w}}{dR}$$
is the water cut rising rate;
f_{w} is the water cut of the oil reservoir;
f_{w0} is the initial water cut of the oil reservoir;
f_{wL} is the water cut limit of the oil reservoir;
R is the degree of recovery of the oil reservoir;
R₀ is the degree of recovery of crude oil when the water cut of the oil reservoir is f_{w0};
E_{R} is the ultimate recovery of crude oil when the water cut of the oil reservoir is the water cut limit f_{wL};
c=ln(10).
